(19) United States
(12) Patent Application Publication
(10) Pub. No.: US 2023/0301846 A1
(43) Pub. Date: Sep. 28, 2023

(54) MATERIALS AND METHODS OF MANUFACTURING RELATED TO APPARATUS FOR COLLECTING URINE

(71) Applicant: ACS Industries, Inc., Lincoln, RI (US)

(72) Inventor: George C. Greenwood, Tiverton, RI (US)

(73) Assignee: ACS Industries, Inc., Lincoln, RI (US)

(21) Appl. No.: 18/187,629

(22) Filed: Mar. 21, 2023

Related U.S. Application Data
(60) Provisional application No. 63/322,249, filed on Mar. 22, 2022.

(51) Int. Cl. A61F 13/537; A61F 13/15

(52) U.S. Cl. CPC A61F 13/53717; A61F 13/15642; A61F 13/15723; A61F 13/15739; A61F 2013/15715; A61F 2013/530364

(57) ABSTRACT

The present disclosure provides improvements related to the following: (1) various materials for use in the external permeable wicking cover (outer bandage covering); (2) the placement of a shape retention element within an edge seam of the core wherein the shape retention element has rolled ends to prevent any sharp ends; and (3) various production improvements related to heat blooming of core for shape retention, continuous processing of the external covering and core, automatic cutting to length during production, vacuum hold for insertion of the core into the outer impermeable housing, and heat mold gluing/forming of the outer covering to the inner core.

### CROSS-REFERENCE TO RELATED APPLICATIONS

This application claims the benefit of U.S. Provisional Application No. 63/322,249, filed Mar. 22, 2022, the entire contents of which is incorporated herein by reference.

### BACKGROUND OF THE DISCLOSURE

The present disclosure relates generally to external urinary collection apparatus. Disabled or injured persons, or persons with restricted movement during long periods of time, i.e. pilots, may have an impaired or limited mobility that prevents them from urinating in a sanitary manner.

External urinary collection apparatus are known in the art. U.S. patent Ser. No. 10/226,376 discloses and describes a well known external urinary collection apparatus which has wide industry acceptance. The entire contents and disclosure of USP10226376 are incorporated herein by reference.

As with all high-volume medical products there are always continuing efforts to improve the product and materials, improve manufacturing techniques, and to reduce costs.

### SUMMARY OF THE DISCLOSURE

The present disclosure provides improvements related to the following:
(1) improved wicking materials for use in the external permeable membrane (outer bandage covering);
(2) the placement of a forming wire within the core wherein the forming wire has rolled ends to prevent any sharp ends; and
(3) various production improvements related to continuous processing of the external covering and core, automatic cutting to length during production, vacuum hold for insertion of the core into the outer impermeable covering, and heat mold gluing/forming of the outer covering to the inner core.

### BRIEF DESCRIPTION OF THE DRAWING FIGURES

Exemplary embodiments will now be described further by way of example with reference to the following examples and figures, which are intended to be illustrative only and in no way limiting upon the scope of the disclosure.

FIG. 1 is a perspective view of an exemplary device in accordance with the teachings of the present invention;

FIG. 2 is an exploded perspective view thereof;

FIG. 3 is a perspective view of the core assembly thereof; and

FIG. 4 is a cross-sectional view thereof taken along line 4-4 of FIG. 3.

### DESCRIPTION OF THE EXEMPLARY EMBODIMENTS

Certain exemplary embodiments will now be described to provide an overall understanding of the principles of the structure, function, manufacture, and use of the device and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Further, to the extent that directional terms like top, bottom, up, or down are used, they are not intended to limit the systems, devices, and methods disclosed herein. A person skilled in the art will recognize that these terms are merely relative to the system and device being discussed and are not universal.

The present disclosure generally provides improvements related to the following features and methods of manufacturing or assembly of a urine collecting device:
(1) improved wicking materials for use in the external permeable membrane (outer bandage covering);
(2) the placement of a forming wire within the core wherein the forming wire may have rolled ends to prevent any sharp ends; and
(3) improved production and assembly methods related to continuous processing of the external covering and core, continuous seaming or sewing of the core assembly, automatic cutting to length during production, vacuum hold for insertion of the core into the outer impermeable covering, and heat mold gluing/forming of the outer housing to the inner core.

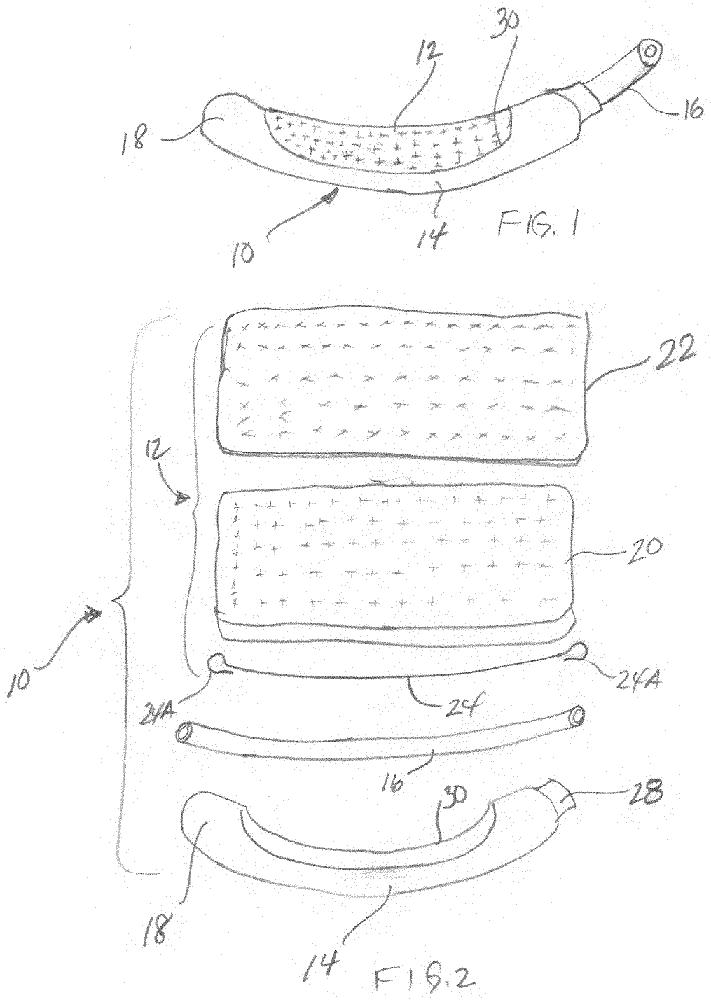

Referring to FIGS. 1 and 2 a device for collecting urine is illustrated and generally indicated at 10. The device 10 comprises a fluid permeable core assembly 12, an elongated capsular-shaped, fluid impermeable housing 14, and a suction tube 16.

In an exemplary use, the urine collection device 10 is arranged in a generally vertical position in contact with or close proximity to the subject's urethra, such that a fluid, such as urine, can be absorbed by the permeable core assembly 12, flow through the permeable core assembly and, by gravity, flow downwardly into a housing reservoir 18 where it can be removed through the suction tube 16. The general construction and function of the device 10 is known and described in USP 10226376 and therefore the broader aspects of the device will not be discussed in detail. The present disclosure focused on several improved material features as well as manufacturing and assembly processes.

Referring to FIGS. 2-4, the permeable core assembly 12 comprises a tubular, fluid permeable core 20 having a cylindrical external surface, a fluid permeable wicking cover 22 disposed on the external surface of the tubular fluid permeable core 20, and may also include an elongated shape retention element 24 extending along a longitudinal extent of the core, the shape retention element 24 being entirely imbedded within the core 20.

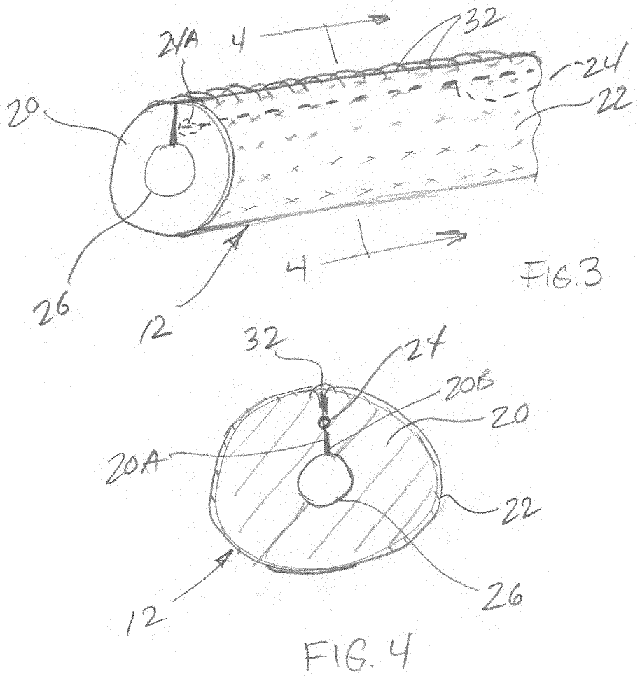

The fluid permeable core 20 may be a non-woven fibrous bio-compatible polymer material (such as non-woven nylon or polyester) which is produced as a flat slab, or bat, of material (batting) (See FIG. 2) with thickness and planar dimensions which will result in, when rolled and seamed at the side edges, a cylindrical core (FIGS. 3-4) having a diameter of approximately 1.0 to 2.5 inches. The rolled material may form an internal axial lumen 26 extending longitudinally through the center of the core.

The fluid permeable wicking cover 22 is disposed on the outer surface of the core 20 and comprises a bio-compatible natural fiber material, which may in some embodiments comprise at least one layer of non-woven bamboo material. In some embodiments, the wicking cover may comprise two-layers of bamboo material (2-ply). Non-woven, natural bamboo fiber material has been tested and shown to provide a superior wicking or capillary action to draw urine away from the user's skin into the core and thus provide improved comfort with less skin irritation.

The shape retention element 24 may comprise a wire or other formed material which may retain a predefined arcuate curvature which is suitable for the intended anatomy of the end user. In this regard, the wire 24 may be pre-shaped with a specific arcuate curvature. In the case of a pre-shaped wire, it is also preferable that the wire ends be rolled over or formed into eyelets 24A so that wire end turned back inwardly and the terminal end of the element has a rounded surface to prevent piercing through the device and injuring the user. To further prevent injury to the user, the shape retention element may be imbedded within the core material 20 during formation of the batted core material or may be inserted and imbedded between the side edges of the core material during seaming of the edges to form the core (see FIGS. 3-4). This process will be described further hereinafter.

The capsular-shaped housing 14 may be molded or formed from a water or fluid impermeable pliable polymer or other pliable material. The housing 14 may have a reservoir 18 at a lower end for collecting a volume of liquid, or urine, that has been wicked inwardly to the core assembly 12 and that has flowed downwardly by gravity towards the lower end of the device 10. The housing 14 may further comprise an outlet port 28 at an upper end thereof through which collected urine may be removed through the suction tube 16.

The housing 14 still further includes an elongated collection window 30 extending at least partially between the lower reservoir end and the upper outlet port end. The core assembly 12 is received within the housing 14 such that the core assembly is disposed within and extends across the elongated window 30.

The suction tube 16 may generally be formed from a flexible PVC or urethane tubing material and be of a sufficient length such that it may extend through the interior lumen 26 of the core assembly 12 with a first end disposed in the reservoir and a second end exiting the housing 14 through the outlet port 28 where it can be attached to a vacuum source (not shown).

One noted improvement of the present device is that the core assembly 12 is adhered to an interior of the housing 14 during assembly. It has been found that in some cases, consumers may be reusing the housings 14 with new core materials to save costs. This is not a recommended activity due to the potential for unsanitary conditions if proper cleaning and disinfecting of the housing is not provided. Adhering the core assembly 12 to the housing 14 prevents the consumer from disassembling the device and replacing the internal components for re-use and creates a more sanitary single-use device.

In some embodiments, the finished core assembly 12 may be placed into a mold and heated to release and reset the polymer core material into a more consistent cylindrical shape which will improve comfort and generally improve consistency in assembly of the core with the housing. In some embodiments, the mold may be provided with an arcuate curvature to not only round the diameter of the core but also provide it with a predefined shape consistent with the arcuate curvature of the shape retention element generally improving the shape retention of the entire device after assembly.

In some embodiments, the shape retention element 24 may be formed from a ductile material which can be bent into a predefined curvature at the time of use.

With respect to production of the device components, an improved method of continuously forming a plurality of core assemblies 12 may comprise the steps of:
providing a continuous length of non-woven permeable core material 20;
providing a continuous length of non-woven permeable wicking cover material 22, wherein the wicking cover material is wider than the core material;
continuously overlaying the wicking cover material 22 over the core material 20;
continuously rolling the overlapped core and cover materials into a tubular shape with opposing side edges 20A/20B aligned in adjacent facing relation and the cover material facing outwardly;
continuously securing the aligned side edges together to form a continuous tubular core assembly; and
repeatedly cutting the continuous core assembly into discrete core assemblies of a predefined length.

In some embodiments, the step of continuously securing the side edges 20A/20B may comprise continuously side stitching the opposing side edges with a bio-compatible polymer thread 32. When using side stitching, the method may comprise the step of flattening the stitched side seam such that the side edges 20A/20B become aligned in face-to-face relation.

In other embodiments, the step of continuously securing the side edges may comprise continuously ultrasonically welding the opposing side edges.

In some embodiments, the method may further comprise the step of placing each cut core assembly in a cylindrical mold and applying heat to the mold to release and reset the core material into a stable cylindrical shape.

An improved method of assembling a urine collecting device to prevent re-use comprises the steps of:
providing a permeable core;
providing an impermeable housing;
placing the housing in a vacuum bed;
applying an adhesive to an inside surface of the housing;
inserting the core into the housing;
placing the assembled core and housing into a mold having a predefined shape; and
applying heat to the mold to adhere the core to the housing thereby preventing removal of the core from the housing.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A device for collecting urine comprising:
a permeable core assembly comprising:
a tubular fluid permeable core having a cylindrical external surface;
a fluid permeable wicking cover disposed on the external surface of the tubular fluid permeable core, the fluid permeable wicking cover comprising at least one layer of non-woven bamboo material; and
an elongated shape retention element extending along a longitudinal extent of the core, the shape retention element being entirely imbedded within the core;
an elongated, capsular-shaped, fluid impermeable housing having a reservoir at a lower end, an outlet port at an upper end, and an elongated window extending at least partially between the lower end and the upper end, the core assembly received within the housing such that the core assembly is disposed within and extends across the elongated window; and
a suction tube extending through an interior of the core assembly and having a first end disposed in the reservoir and a second end exiting the housing through the outlet port,
wherein an exterior of the core is adhered to an interior of the housing.

2. The device of claim 1 wherein the wicking cover comprises two layers.

3. The device of claim 1 wherein the shape retention element is formed with a predefined curvature.

4. The device of claim 1 wherein core assembly is formed with a predefined curvature.

5. The device of claim 1 wherein the shape retention element comprises a ductile metal and is bendable to a predefined shape.

6. The device of claim 1 wherein the core comprises a rectangular slab of permeable material which is rolled into a tubular form, with opposing side edges thereof secured together to form a tube.

7. The device of claim 6 wherein the opposing side edge are sewn together with a longitudinal seam stitch and the shape retention element is imbedded between the opposing side edges during stitching.

8. The device of claim 1 wherein the core comprises a rectangular slab of permeable material which is rolled into a tubular form, with opposing side edges thereof secured together to form a tube.

9. The device of claim 6 wherein the opposing side edge are sewn together with a longitudinal seam stitch and the shape retention element is imbedded between the opposing side edges during stitching.

10. A method of continuously forming a plurality of core assemblies for a urine collection device comprising the steps of:
providing a continuous length of non-woven permeable core material;
providing a continuous length of non-woven permeable wicking cover material, wherein the wicking cover material is wider than the core material;
continuously overlaying the wicking cover material over the core material;
continuously rolling the overlapped core and cover materials into a tubular shape with opposing side edges aligned in adjacent facing relation and the cover material facing outwardly;
continuously securing the aligned side edges together to form a continuous tubular core assembly; and
repeatedly cutting the continuous core assembly into discrete core assemblies of a predefined length.

11. The method of claim 10 wherein the step of continuously securing comprises continuously side stitching the opposing side edges.

12. The method of claim 11 further comprising the step of flattening the stitched side seam.

13. The method of claim 10 wherein the step of continuously securing comprises the step of continuously ultrasonically welding the opposing side edges.

14. The method of claim 10 further comprising the step of placing each core assembly in a cylindrical mold and applying heat to the mold to release and reset the core material into a stable cylindrical shape.

15. The method of claim 12 further comprising the step of placing each core assembly in a cylindrical mold and applying heat to the mold to release and reset the core material into a stable cylindrical shape.

16. The method of claim 13 further comprising the step of placing each core assembly in a cylindrical mold and applying heat to the mold to release and reset the core material into a stable cylindrical shape.

17. A method of assembling a urine collecting device comprising the steps of:
providing a permeable core;
providing a capsular-shaped impermeable housing;
placing the housing in a vacuum bed;
applying an adhesive to an inside surface of the housing;
inserting the core into the housing;
placing the assembled core and housing into a mold having a predefined shape; and
applying heat to the mold to adhere the core to the housing thereby preventing removal of the core from the housing.
